(19) United States
(12) Patent Application Publication
(10) Pub. No.: US 2017/0105937 A1
(43) Pub. Date: Apr. 20, 2017

(54) OLAPARIB CO-PRECIPITATE AND PREPARATION METHOD THEREOF

(71) Applicant: CADILA HEALTHCARE LIMITED, Gujarat (IN)

(72) Inventors: Shafiq un Nabi SHEIKH, Gujarat (IN); Mukesh UKAWALA, Gujarat (IN); Tishir GANDHI, Gujarat (IN); Brijesh Kumar SRIVASTAVA, Gujarat (IN); Muthaiyyan Essakimuthu KANNAN, Gujarat (IN); Ranjit C. DESAI, Gujarat (IN)

(21) Appl. No.: 15/295,235

(22) Filed: Oct. 17, 2016

(30) Foreign Application Priority Data
Oct. 16, 2015 (IN) 3937/MUM/2015

(51) Int. Cl. A61K 9/14; A61K 47/38; A61K 31/502

(52) U.S. Cl. CPC A61K 9/146; A61K 31/502; A61K 47/38

(57) ABSTRACT

The present invention relates to co-precipitates of olaparib and an ionic polymer and pharmaceutical composition containing the co-precipitates. Further, the present invention relates to a method of treating disorders in a patient in need thereof, comprising administering a therapeutically effective amount of said composition.

### FIELD OF THE INVENTION

The present invention relates to pharmaceutical compositions comprising olaparib. In particular, the present invention relates to pharmaceutical compositions comprising a co-precipitate comprising olaparib and an ionic polymer. The invention also relates to processes for the preparation of such compositions. The invention also relates to a method of treating disorders, wherein inhibition of polyADPribosepolymerase (PARP) is desired.

### BACKGROUND OF THE INVENTION

The following discussion of the prior art is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

Olaparib is an FDA-approved targeted therapy for cancer. It is a PARP inhibitor, inhibiting polyADPribosepolymerase (PARP), an enzyme involved in DNA repair. It acts against cancers in people with hereditary BRCA1 or BRCA2 mutations, which, include some ovarian, breast, and prostate cancers. It is marketed in the form of capsules under the trade names Lynparza® in the United States and is chemically known as 4-[3-(4-cyclopropanecarbonyl-piperazine-1-carbonyl)-4-fluoro-benzyl]-2H-phthalazin-1-one and has the following chemical structure of Formula (I),

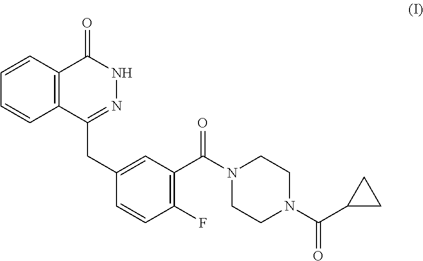 [SMILES: O=C(c1cc(Cc2n[nH]c(=O)c3ccccc23)ccc1F)N1CCN(C(=O)C2CC2)CC1]

Olaparib is essentially neutral across the physiological pH range and belongs to class 4 within the Biopharmaceutics Classification System (BCS) because of its poor solubility and moderate permeability. Due to low solubility in water it has a low dissolution rate and as a result exhibits poor bioavailability.

U.S. Pat. No. 8,475,842 discloses oral formulation of olaparib containing solid dispersion of olaparib in copovidone, prepared by hot-melt extrusion method, to improve its bioavailability. The method of producing the solid dispersion involves mixing of olaparib with copovidone, raising the temperature of the mixture to produce a melt and extrusion of the melt to produce a solid dispersion.

Chinese Publication No. 104434809 discloses oral formulation of olaparib containing solid dispersion of olaparib in povidone, prepared by hot-melt extrusion method and solvent evaporation method.

The main drawback of hot-melt extrusion method is that it tends to lead to drug decomposition due to the high temperatures required to melt the polymer. Additionally, the solid dispersion method carry inherent limitations concerning physical stabilities of the solid dispersion on storage, as it contains amorphous form of active ingredient having improved solubility in water as compared to the crystalline form, but less stability due to its tendency to crystallize.

Therefore, it would be desirable to develop a technique which alleviates the above described disadvantages associated with the known techniques and which provides a co-precipitate of olaparib in amorphous form, which is stable during stability and doesn't convert to crystalline form.

### SUMMARY OF THE INVENTION

In one general aspect, there is provided a co-precipitate comprising olaparib and one or more ionic polymers.

In another general aspect, there is provided a stable amorphous co-precipitate comprising olaparib and one or more ionic polymers.

In another general aspect, there is provided a process for preparing a co-precipitate of olaparib and one or more ionic polymer.

In another general aspect, there is provided a process of preparing a co-precipitate comprising olaparib and ionic polymer, the process comprising:

(a) providing a solution of olaparib and one or more ionic polymer in one or more solvents to form a solution;
(b) co-precipitating the olaparib and ionic polymer from the solution; and
(c) obtaining the co-precipitates by the removal of the solvents.

In another general aspect, there is provided a pharmaceutical composition comprising a co-precipitate of olaparib and one or more ionic polymer.

Embodiments of the pharmaceutical composition may include one or more of the following features. For example, the composition may further include one or more pharmaceutically acceptable excipients selected from diluents, fillers, solubilizers, binders, disintegrants, lubricants, and glidants. Olaparib may be present in a therapeutically effective amount.

The details of one or more embodiments of the invention are set forth in the description below. Other features, objects and advantages of the invention will be apparent from the description.

### BRIEF DESCRIPTION OF DRAWINGS

FIG. 1: XRPD pattern of co-precipitate of olaparib and hypromellose acetate succinate obtained in Example 1.

### DETAILED DESCRIPTION OF THE INVENTION

The above and other objects of the present invention are achieved by the process of the present invention, which leads to greater stability of amorphous co-precipitate of olaparib and ionic polymer. It has now surprisingly been found that composition containing co-precipitate of olaparib with ionic polymer gives excellent formulation characteristics in the form of improved solubility and storage stability. The amorphous co-precipitate has been found to be stable under stability conditions.

As used herein, the term “ionic polymer” includes both anionic (negatively charged) and cationic (positively charged) polymers.

As used herein, the term “co-precipitation” defines the process of precipitating two or more components together from solution.

As used herein, the term “co-precipitate” means any solid composition having at least two components. In certain embodiments, a co-precipitate as disclosed herein includes olaparib co-precipitated with at least one other component, for example a polymer.

As used herein the term “immobilize” with reference to the immobilization of the olaparib in the polymer matrix, means that molecules of the olaparib interact with molecules of the polymer in such a way that the molecules of the olaparib are held in the aforementioned matrix and prevented from crystal nucleation due to lack of mobility.

As used herein, the term “therapeutically effective” indicates that the materials or amount of material is effective to prevent, alleviate, or ameliorate one or more symptoms of a disease or medical condition, and/or to prolong the survival of the subject being treated. A therapeutically-effective amount of olaparib may vary according to disease state, age and weight of the subject being treated. The therapeutically effective amount of olaparib could be from 10 mg to 1000 mg once or twice a day.

As used herein, the term “stable co-precipitate” comprises an amorphous olaparib, measured by an absence of conversion of the amorphous form of olaparib to a crystalline form of olaparib and free from residual solvents after the amorphous olaparib is exposed to a relative humidity of 75% at 40° C. or 60% at 25° C. for a period of at least three months.

In general, the term “free from residual solvents” herein means residual solvents are within the permissible ICH limits suitable for pharmaceutical preparations. For example but not limited to less than 0.5%, particularly less than 0.3% or more particularly less than 0.2%, or most particularly not in detectable amount.

In one general aspect, there is provided a co-precipitate comprising olaparib and one or more ionic polymers.

In general, the ionic polymers include one or more of hydroxypropylmethyl cellulose acetate succinate, hydroxypropylmethyl cellulose phthalate, and methacrylic acid copolymers. In particular, the polymer is hydroxypropylmethyl cellulose acetate succinate.

In general, the methacrylic acid copolymers as used herein may include, but not limited to, methacrylic acid copolymers, methacrylic acid-methacrylate copolymers, methacrylic acid-ethyl acrylate copolymers, ammonium methacrylate copolymers, and aminoalkyl methacrylate copolymers. Examples of methacrylic acid copolymers include Eudragit L100-55, Eudragit 30D-55, Eudragit-L100, Eudragit S100, and Eudragit P-4135F.

In one embodiment, the co-precipitate of the present invention comprises olaparib dispersed within a matrix formed by an ionic polymer such that it is immobilized in its amorphous form. The ionic polymer may prevent intramolecular hydrogen bonding or weak dispersion forces between two or more drug molecules of olaparib. The co-precipitate provides for a large surface area allowing for improved dissolution and bioavailability of olaparib.

In another embodiment, the ratio of the amount by weight of olaparib within the co-precipitate to the amount by weight of the ionic polymer therein is from about 1:9 to about 1:0.5. In particular, the ratio of the amount by weight of olaparib within the co-precipitate to the amount by weight of the ionic polymer therein is from about 2:8 to about 4:6. More particularly, the ratio of the amount by weight of olaparib within the co-precipitate to the amount by weight of the ionic polymer therein is about 1:3.

In another embodiment, olaparib may be present in the co-precipitate in an amount of from about 10% to about 80%, by weight, of the co-precipitate; or in an amount of from about 20% to about 60%, by weight, of the co-precipitate; or in an amount of about 30%, by weight, of the co-precipitate.

In another embodiment, the polymer may be present in the co-precipitate in an amount of not less than about 20%, by weight, of the co-precipitate; or in an amount of from about 20% to about 90% by weight of the co-precipitate; or in an amount of from about 40% to about 80% by weight of the co-precipitate.

In one embodiment, there is provided the process that includes one or more of the following features. For example, the co-precipitate of olaparib and ionic polymer may be prepared by solvent controlled co-precipitation or by spray drying method or lyophilization.

In another general aspect there is provided a process of preparing a co-precipitate comprising olaparib and ionic polymer, the process comprising:

(a) providing a solution of olaparib and one or more ionic polymers in one or more solvents to form a solution;
(b) co-precipitating the olaparib and ionic polymer from the solution; and
(c) obtaining the co-precipitates by the removal of the solvents.

In general, the solvent comprises one or more of dimethylacetamide, dimethyl sulfoxide, dimethylformamide, N-methylpyrrolidone or mixture thereof. In particular, the solvent is dimethylacetamide.

In general, the co-precipitates are precipitated from the solution of step (a) containing olaparib and ionic polymer by combining the solution with an aqueous phase. The pH of the aqueous phase was adjusted to acidic. In particular, the pH is less than 7, more particularly, the pH is less than 3.

In general, the process comprises combining the solution containing olaparib and ionic polymer with the aqueous phase resulting in the co-precipitation of olaparib and ionic polymer. The co-precipitates obtained as a suspension containing particles of olaparib and ionic polymer.

In general, the removal of the solvent comprises one or more of evaporation by rotational distillation, evaporation under reduced pressure, spray drying, agitated thin film drying (“ATFD”), freeze drying (lyophilization), flash evaporation, filtration, decantation, centrifugation and vacuum distillation thereby leaving the amorphous co-precipitate in a matrix formed by the polymer. In particular, the co-precipitate of olaparib and ionic polymer is obtained by removal of the solvents comprising one or more of conventional filter techniques or centrifuges. The suspension may be centrifuged and washed with water several times to remove the residual solvent. The obtained wet-cake is then dried to get co-precipitates of the present invention.

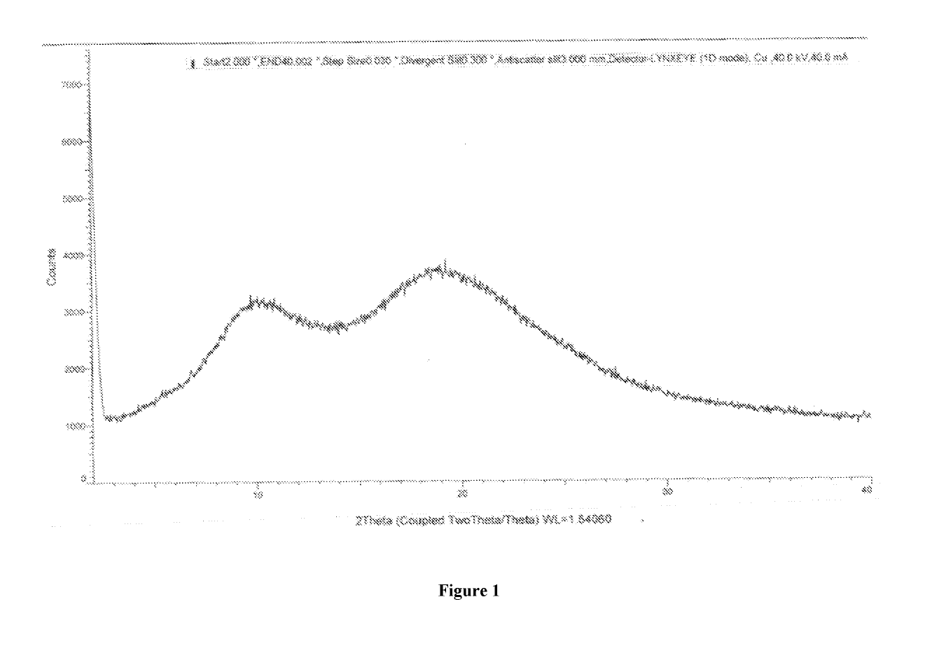

In general, the co-precipitates of the invention is amorphous characterized by X-ray powder diffraction pattern substantially as depicted in FIG. 1.

In general, the amorphous co-precipitate of olaparib and ionic polymer is stable. In one embodiment, olaparib in the form of co-precipitate of the present invention retains amorphous form while being exposed to a relative humidity of 75% at 40° C. or 60% at 25° C. for a period of at least three months.

In another general aspect there is provided a pharmaceutical composition comprising a co-precipitate of olaparib and one or more ionic polymers.

In general, the pharmaceutical composition further comprises one or more pharmaceutically acceptable excipients.

In general, the pharmaceutical compositions comprising co-precipitate of olaparib of the invention may be prepared by using diluents or excipients such as fillers, bulking agents, binders, wetting agents, disintegrating agents, surface active agents, and lubricants. Various modes of administration of the pharmaceutical compositions of the invention can be selected depending on the therapeutic purpose, for example tablets, pills, powders, liquids, suspensions, emulsions, granules, capsules, suppositories, or injection preparations.

Various additives can be mixed with the co-precipitates as described herein to form a material suitable for the above dosage forms. The additives may fall generally into the following classes: diluents, solubilizers, disintegrants, binders, and lubricants.

In general, the diluents may include, but not limited to, lactose, mannitol, xylitol, microcrystalline cellulose, calcium diphosphate, and starch.

In general, the disintegrants may include, but not limited to, sodium starch glycolate, sodium alginate, carboxymethyl cellulose sodium, methyl cellulose, and croscarmellose sodium.

In general, the binders may include, but not limited to, methyl cellulose, microcrystalline cellulose, starch, and gums such as guar gum, and tragacanth.

In general, the lubricants may include, but not limited to, stearic acid and its derivatives or esters like sodium stearate, magnesium stearate and calcium stearate, talc, and colloidon silicon dioxide.

In another general aspect, there is provided a pharmaceutical dosage form comprising co-precipitates comprising therapeutically effective amount of olaparib as described herein for use in therapy, in particular for use in the treatment of cancer, for example breast or ovarian cancer, and particularly cancers for which inhibition of Poly(ADP-ribose) polymerase (PARP) is required.

In another general aspect, there is provided a method of treating cancer by administering a pharmaceutical dose of olaparib in a patient in need of said drug, comprising administering to said patient a formulation comprising co-precipitates of olaparib and ionic polymer, wherein the dose comprises 10 to 1000 mg of olaparib in form of co-precipitate with an ionic polymer.

In another general aspect there is provided method of treating cancer by administering a pharmaceutical dose of olaparib in a patient in need comprising administering to said patient a formulation comprising co-precipitates of olaparib and one or more ionic polymers.

Powder X-ray Diffraction: X-ray powder diffraction spectrum was observed on a X-ray Powder diffractometer of make Rigaku or PANanalytical or equivalent make having a Copper Kα-radiation at a voltage of 40 kV and 30 mA. Approximately 150 mg sample was gently flattened on a quartz plate without further processing (e.g. Grinding and sieving) and scanned from 4° to 40° at 0.010° sampling width and 4.000° per minute.

The invention is further illustrated by the following examples which are provided merely to be exemplary of the invention and do not limit the scope of the invention. Certain modification and equivalents will be apparent to those skilled in the art and are intended to be included within the scope of the present invention.

### EXAMPLES

### Example 1

### Preparation of Capsule Containing Co-Precipitates of Olaparib and Hypromellose Acetate Succinate

In a 250 mL three necked round bottom flask equipped with nitrogen atmosphere facility, mechanical stirrer, thermometer and an addition funnel, olaparib (2.5 g) and hypromellose acetate succinate (7.5 g) were dissolved in dimethylacetamide (23.3 g) at 40° to 50° C. 0.01 N HCl solution (233.3 ml) was added and stirred. The resulting suspension containing particles of solid dispersion of olaparib in hypromellose acetate succinate was filtered to obtain co-precipitates of Olaparib and hypromellose acetate succinate. The wet-cake was washed with 0.01 N HCl solution and water, dried to obtain co-precipitates of olaparib with hypromellose acetate succinate. The obtained dry olaparib premix was calculated on the basis of its assay content and dispensed into to a fill weight of 250 mg per capsule using manual capsule filling machine.

### Example 2

### Preparation of Tablet Containing Co-Precipitates of Olaparib and Hypromellose Acetate Succinate

| Ingredients | Amount (mg) | % w/w |
|---|---|---|
| Olaparib-Hypromellose | 200.0 mg | 50.0% |
| acetate succinate (premix) | (containing 50.0 mg of Olaparib) | |
| Microcrystalline cellulose | 174.0 mg | 43.5% |
| Croscarmellose sodium | 16.0 mg | 4.0% |
| Magnesium stearate | 4.0 mg | 1.0% |
| Colloidal silica | 6.0 mg | 1.5% |

Immediate release tablets were prepared using direct compression method. Olaparib-hypromellose acetate succinate premix, microcrystalline cellulose, croscarmellose sodium, magnesium stearate and colloidal silica were weighed and mixed together.

The blended material was sieved through a 40 mesh sieve, compressed to make a tablet formulation using tablet compression machine.

### Example 3

### Preparation of Co-Precipitates of Olaparib and Eudragit L100-55

In a 250 mL three necked round bottom flask equipped with nitrogen atmosphere facility, mechanical stirrer, thermometer and an addition funnel, olaparib (2.5 g) and Eudragit L100-55 (7.5 g) were dissolved in dimethylacetamide (23.3 g) at 40° to 50° C. 0.01 N HCl solution (233.3 ml) was added and stirred. The resulting suspension containing particles of co-precipitates of olaparib in Eudragit L100-55 was filtered to obtain co-precipitates of Olaparib and Eudragit L100-55. The wet cake was washed with 0.01 N HCl solution and water, dried to obtain co-precipitates of olaparib with Eudragit L100-55. The co-precipitates were used as drug-polymer premix for further use in making suitable solid oral dosage form.

### Example 4

### Preparation of Tablet Containing Co-Precipitates of Olaparib and Eudragit L100-55

| Ingredients | Amount (mg) | % w/w |
|---|---|---|
| Olaparib-Eudragit L100-55 | 200.0 mg | 50.0% |
| (premix) | (containing 50.0 mg of Olaparib) | |
| Microcrystalline cellulose | 174.0 mg | 43.5% |
| Croscarmellose sodium | 16.0 mg | 4.0% |
| Magnesium stearate | 4.0 mg | 1.0% |
| Colloidal silica | 6.0 mg | 1.5% |

Immediate release tablets were prepared using direct compression method. Olaparib-Eudragit L100-55 premix, microcrystalline cellulose, croscarmellose sodium, magnesium stearate and colloidal silica were weighed and mixed together. The blended material was sieved through a 40 mesh sieve, compressed to make a tablet formulation using tablet compression machine.

### Reference Example 1

### Preparation of Conventional Immediate Release Capsule Containing Olaparib

| Ingredient | mg/capsule | % w/w |
|---|---|---|
| Olaparib | 50.00 | 25.00 |
| Lactose | 119.00 | 59.50 |
| Micro crystalline cellulose | 20.00 | 10.00 |
| Croscarmellose Na | 8.00 | 4.00 |
| Sodium Lauryl Sulphate | 1.00 | 0.50 |
| Magnesium stearate | 2.00 | 1.00 |
| Net ingredients weight | 200.00 | 100.00 |

Lactose, Microcrystalline cellulose, Croscarmellose Na, Sodium Lauryl Sulphate and Magnesium stearate were weighed, sifted with 30# sieve and mixed properly. This blend was further mixed with Olaparib and filled into capsules.

### Reference Example 2

### Preparation of Capsule Containing Solid Dispersion of Olaparib and Lauryl Macrogolglyceride

| Ingredient | mg/capsule | % w/w |
|---|---|---|
| Olaparib | 50.00 | 10.00 |
| Lauryl Macrogolglyceride | 450.00 | 90.00 |
| (Gelucire 44/14 grade) | | |

Lauryl macrogolglyceride was melted at about 50° to 70° C. into a vessel. To this olaparib was added and the contents mixed to achieve a homogeneous suspension. A 500 mg of melted dispersion was filled in gelatin capsule.

### Example 5

### In Vitro Dissolution of Olaparib Preparations

Dissolution was performed according to the general procedure of the Unites States Pharmacopeia Apparatus-I (Basket). Media used for dissolution was 6.8 pH Phosphate buffer, temperature 37±0.5° C. and rotate 50 rpm. The results of this test are shown in Table I.

TABLE 1
In Vitro dissolution of Olaparib preparations

| | Amount of Olaparib Released (%) | | | |
|---|---|---|---|---|
| Sample | 15 min | 30 min | 45 min | 60 min |
| Example 1 | 36 | 58 | 69 | 75 |
| Reference Example-1 | 31 | 42 | 48 | 53 |
| Reference Example-2 | 17 | 27 | 31 | 33 |

While the invention has been described in terms of its specific embodiments, certain modifications and equivalents will be apparent to those skilled in the art and are intended to be included within the scope of the invention.

We claim:

1. A co-precipitate comprising olaparib and one or more ionic polymer.

2. The co-precipitate according to claim 1, wherein the ionic polymer comprises one or more of hydroxypropylmethyl cellulose acetate succinate, hydroxypropylmethyl cellulose, hydroxypropyl methyl cellulose phthalate and methacrylic acid copolymers.

3. The co-precipitate according to claim 1, wherein the ionic polymer is hydroxypropylmethyl cellulose acetate succinate.

4. The co-precipitate according to claim 1, wherein the olaparib is present in amorphous form.

5. The co-precipitate according to claim 1, wherein the ratio of olaparib to ionic polymer is from about 1:9 to about 1:1.

6. A process of preparing a co-precipitate comprising olaparib and ionic polymer, the process comprising:
(a) providing a solution of olaparib and one or more ionic polymers in one or more solvents to form a solution;
(b) co-precipitating the olaparib and the ionic polymer from the solution; and
(c) obtaining the co-precipitates by the removal of the solvents.

7. The process according to claim 6, wherein the solvent in step (a) comprises one or more of dimethylformamide, dimethylacetamide, dimethyl sulfoxide, and N-methyl pyrrolidone.

8. The process according to claim 6, wherein the olaparib and the ionic polymer are co-precipitated by combining solution of step (a) with an aqueous phase.

9. The process according to claim 6, wherein the removal of solvent in step (c) comprises one or more steps selected from evaporation by rotational distillation, evaporation under reduced pressure, spray drying, agitated thin film drying, freeze drying, flash evaporation, filtration, decantation, centrifugation and vacuum distillation.

10. A pharmaceutical composition comprising a co-precipitate of olaparib and one or more ionic polymers.

11. The pharmaceutical composition according to claim 10, further comprising a pharmaceutically acceptable carrier.

12. A method of treating cancer by administering a pharmaceutical dose of olaparib in a patient in need comprising administering to said patient a formulation comprising co-precipitates of olaparib and one or more ionic polymers.
